(19) United States
(12) Patent Application Publication
(10) Pub. No.: US 2007/0225248 A1
(43) Pub. Date: Sep. 27, 2007

(54) Oral dosage forms of gemcitabine derivatives

(75) Inventors: Finn Myhren, Porsgrunn (NO); Marit Liland Sandvold, Porsgrunn (NO); Ole Henrik Eriksen, Oslo (NO)

Correspondence Address: FITZPATRICK CELLA HARPER & SCINTO, 30 ROCKEFELLER PLAZA, NEW YORK, NY, 10112, US

(73) Assignee: Clavis Pharma AS, Oslo (NO)

(21) Appl. No.: 11/384,270

(22) Filed: Mar. 21, 2006

(51) Int. Cl. A61K 31/7072

(52) U.S. Cl. 514/49

(57) ABSTRACT

The present invention relates to oral dosage forms of certain long chain saturated and monounsaturated fatty acid derivatives of 2′,2′-difluorodeoxycytidine (Gemcitabine). In particular, the present invention relates to the use of the said gemcitabine derivatives or a pharmaceutical acceptable salt thereof for preparing an oral dosage form ameliorating compliance in treatment of cancer.

The present invention relates to oral dosage forms of certain long chain saturated and monounsaturated fatty acid derivatives of 2′,2′-difluorodeoxycytidine (Gemcitabine). In particular, the present invention relates to the use of the said gemcitabine derivatives or a pharmaceutical acceptable salt thereof for preparing an oral dosage form ameliorating compliance in treatment of cancer.

Gemcitabine has the formula:

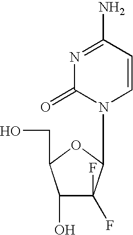

The derivatives of the present invention can be represented by the formula I: 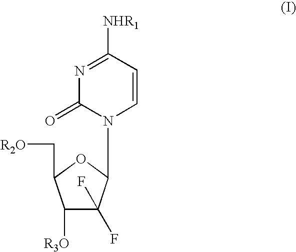
wherein R₁, R₂and R₃ are independently selected from hydrogen and C₁₈— and C₂₀-saturated and monounsaturated acyl groups, with the proviso that R₁, R₂ and R₃ cannot all be hydrogen.

It is known from WO 98/32762 that compounds of formula (I) are useful in treatment of cancer.

Furthermore, gemcitabine is a well known cytostatic compound, marketed under the trade name Gemzar by Eli Lilly & Co.

Gemzar is administered intravenously (i.v.). The reason for choosing a parenteral administration route is due to the toxicity of gemcitabine. Like a lot of drugs, it obviously would have been desirable to be able to administer gemcitabine orally. For the patient oral administration usually is much more pleasant than intravenous administration.

Normally the dose in terms of mg/kg must be increased when administering enterally (orally) compared to parenterally due to bioavailability less than 100%. Therefore, drugs having a high degree of toxicity are not suitable for oral administration.

This is also the case for gemcitabine. Experiments have shown that the toxicity of gemcitabine is greatly enhanced after oral administration. That is, the toxicity of gemcitabine is largely increased after oral administration compared to the toxicity after intraperitoneal (parenteral) administration.

We have now surprisingly found that the toxicity after oral administration of derivatives of formula (I) resembles the toxicity of intraperitoneal (parenteral) dosing of the said compound.

It is a main object of the present invention to find a way to be able to orally administer gemcitabine derivatives being as efficacious as, or more efficacious than gemcitabine itself, in the treatment of cancer.

This and other objects by the present invention are obtained by the attached claims.

According to an embodiment of the present invention the use of a gemcitabine derivative of formula (I): 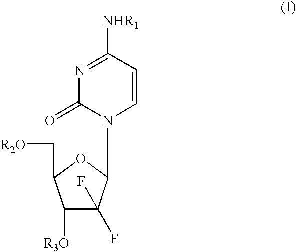
wherein R₁, R₂ and R₃ are independently selected from hydrogen and C₁₈— and C₂₀-saturated and monounsaturated acyl groups, with the proviso that R₁, R₂ and R₃ cannot all be hydrogen or a pharmaceutical acceptable salt thereof, for preparing an oral dosage form ameliorating compliance in treatment of cancer, is provided.

Gemcitabine has three derivatisable functions, namely the 5′- and 3′-hydroxyl groups and the N⁴-amino group. Each group can selectively be transformed into an ester or amide derivative, but di-adducts (di-esters or ester-amides) and tri-adducts may be formed as well. In the case of the di- and tri-adducts the acyl substituent groups need not necessarily be the same.

Currently, the mono-acyl derivatives of this invention, i.e. with two of R₁, R₂ and R₃ being hydrogen, are preferred. It is especially preferred that the monosubstitution with the acyl group should be in the 3′-O and 5′-O positions of the sugar moiety, with 5′-O substitution being most preferred.

The double bond of the mono-unsaturated acyl groups may be in either the cis or the trans configuration, although the therapeutic effect may differ depending on which configuration is used.

The position of the double bond in the monounsaturated acyl groups also seem to affect the activity. Currently, we prefer to use esters or amides having their unsaturation in the ω-9 position. In the ω-system of nomenclature, the position ω of the double bond of a monounsaturated fatty acid is counted from the terminal methyl group, so that, for example, eicosenoic acid (C₂₀:1 ω-9) has 20 carbon atoms in the chain and a single double bond is formed between carbon 9 and 10 counting from the methyl end of the chain. We prefer to use esters, ester-amides and amides derived from oleic acid (C₁₈:1 ω-9, cis), elaidic acid (C₁₈:1 ω-9, trans), eicosenoic acid(s) (C₂₀:1 ω-9, cis) and (C₂₀:1 ω-9, trans), and the amides and 5′-esters are currently the most preferred derivatives of this invention.

Esters, ester-amides and amides of gemcitabine derived from stearic acid (C₁₈:0) and eicosanoic acid (C₂₀:0) are advantageously used in some cases.

Elaidic acid (N⁴)-Gemcitabine amide, elaidic acid (5′)-gemcitabine ester and elaidic acid (3′)-gemcitabine ester among the most preferred derivatives of the invention.

In a preferred embodiment of the invention the use of elaidic acid (5′)-gemcitabine ester for preparing an oral dosage form ameliorating compliance in treatment of cancer, is provided.

According to another embodiment, the present invention relates to an oral dosage form useful for ameliorating compliance in treatment of cancer, comprising a gemcitabine derivative of formula (I) or a pharmaceutical acceptable salt thereof.

The present invention also provides a method for ameliorating compliance in treatment of cancer, in a subject in need of such treatment, which comprises orally administering to such subject a therapeutically effective amount of a gemcitabine derivative of formula (I) as defined in claim 1 or a pharmaceutical acceptable salt thereof.

The derivatives of formula (I) are prepared according to methods known in the prior art (see WO 98/32762 for further details).

The term “therapeutically effective amount” as used herein refers to from about 0.1 mg to 20 grams per day of a gemcitabine derivative of formula (I) or a pharmaceutical acceptable salt thereof, more preferred from about 100 mg to 2 grams per day of a gemcitabine derivative of formula (I) or a pharmaceutical acceptable salt thereof, in a formulation containing 0.001-100% of the said derivative or salt thereof formulated in capsule, tablet, mixture, colloidal suspension or others for oral administration.

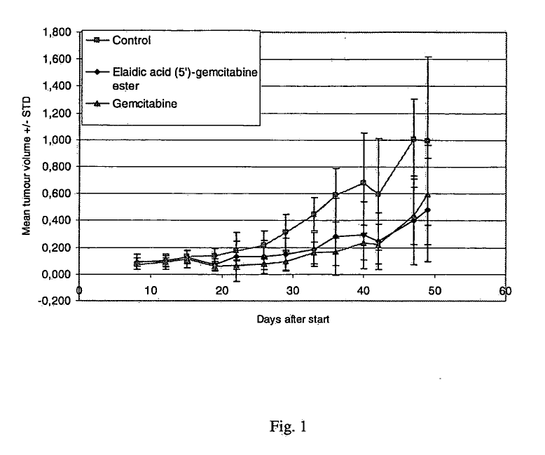

In the following the invention will be further explained by examples and attached figures (FIG. 1-4). The examples are only meant to be illustrative and shall not be considered as limiting.

FIG. 1 shows antitumour activity of elaidic acid (5′)-gemcitabine ester and gemcitabine in colon cancer xenograft Co5776.

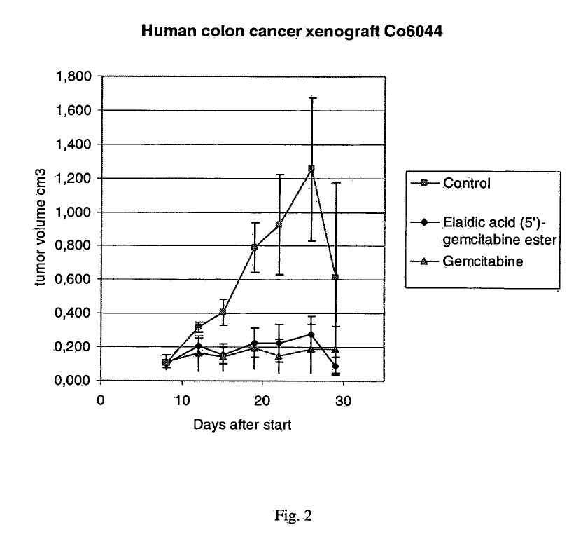

FIG. 2 shows antitumour activity of elaidic acid (5′)-gemcitabine ester and gemcitabine after intraperitoneal administration to mice with human colon cancer xenograft Co6044.

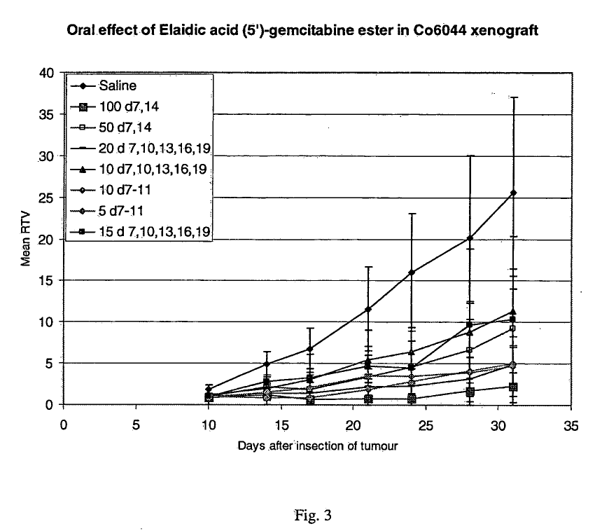

FIG. 3 shows oral effect of elaidic acid (5′)-gemcitabine ester in Co6044 xenograft.

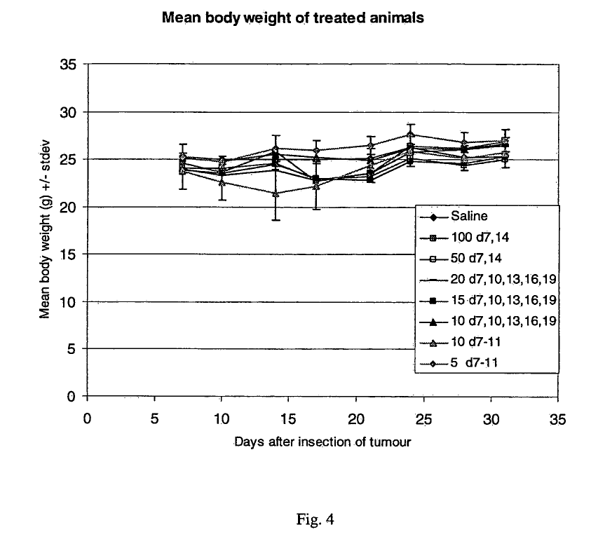

FIG. 4 shows mean body weight of treated animals.

### EXAMPLES

### Example 1

Background Experiments

When test compounds are administered every third day, repeated five times, both test compounds at their maximum tolerated doses (MTD), the maximum tolerated dose for gemcitabine is approximately 120 mg/kg per injection compared to 40 mg/kg per injection for elaidic acid (5′)-gemcitabine ester. This is shown below by the experiments presented in table 1 and table 2 using different mice strains and also different human colon xenografts.

Antitumor Activity of Elaidic Acid (5′)-Gemcitabine Ester and Gemcitabine in a Human Colon Xenograft Model Co5776

Human colon cancer Co5776 was inserted to Ncr:nu/nu female mice subcutaneously, and treatment started when tumors reached a mean volume of 100 mm³. Treatment was IP with gemcitabine (120 mg/kg) or elaidic acid (5′)-gemcitabine ester (40 mg/kg). As can be seen from FIG. 1, high antitumour activity in terms of reductions in tumor growth is obtained for both gemcitabine and elaidic acid (5′)-gemcitabine ester. Toxicity in terms of weight loss is similar, with slightly more toxicity seen with gemcitabine but both are considered to be at the maximum tolerated dose.

TABLE 1
Antitumour activity in NCR: nu/nu female mice implanted with Colon 5776 (human colon
carcinoma) treated IP with elaidic acid (5′)-gemcitabine ester or gemcitabine

| | No. | | | Dose | Toxic | BWC¹ | Optimum |
|---|---|---|---|---|---|---|---|
| Compound | mice | Treatment days | Route | mg/kg | deaths (d) | [%] | T/C [%] |
| Saline | 8 | D8, 11, 14, 17, 20 | IP | | | 1 | |
| Elaidic acid (5′)- | 8 | D8, 11, 14, 17, 20 | IP | 40 | 0 | −4 | 17* |
| gemcitabine ester | | | | | | | |
| Gemcitabine | 8 | D8, 11, 14, 17, 20 | IP | 120 | 1(18) | −5 | 17* |

*significant different from Saline control
¹BWC = body weight change, T/C = volume of treated tumour versus volume of control tumour

Antitumor Activity of Elaidic Acid (5′)-Gemcitabine Ester and Gemcitabine in Human Colon Cancer Xenograft Model

Ncr:nu/nu female mice, 8 per group, were inserted with the human colon cancer xenograft Co6044 and treated IP every third day for five times with elaidic acid (5′)-gemcitabine ester (40 mg/kg) or gemcitabine (120 mg/kg). Treatment started when the tumors reached a mean volume of 100 mm³. Excellent antitumor effect was obtained for elaidic acid (5′)-gemcitabine ester and gemcitabine.

TABLE 2
Antitumour activity in NMRI male mice implanted with Co6044 (human colon carcinoma)
treated IP with elaidic acid (5′)-gemcitabine ester or gemcitabine

| | No. | | | Dose | Toxic | BWC¹ | Optimum |
|---|---|---|---|---|---|---|---|
| Compound | mice | Treatment days | Route | mg/kg | deaths (d) | D 8-15 | T/C [%] |
| Saline | 8 | D8, 11, 14, 17, 20 | IP | | | −1 | |
| Elaidic acid (5′)- | 8 | D8, 11, 14, 17, 20 | IP | 40 | 0 | −1 | 19* |
| gemcitabine ester | | | | | | | |
| Gemcitabine | 8 | D8, 11, 14, 17, 20 | IP | 120 | 0 | −3 | 15* |

*significant different from Saline control
¹BWC = body weight change, T/C = volume of treated tumour versus volume of control tumour

### Example 2

Antitumour Activity of Elaidic Acid (5′)-Gemcitabine Ester and Gemcitabine in Co6044 After Oral Administration

Antitumour activity after oral administration of elaidic acid (5′)-gemcitabine ester and gemcitabine was tested for the first time in NCR:nu/nu mice. The lowest starting dose was selected based on IP data. A dose of gemcitabine that is well tolerated and active when administered intraperitoneally (120 mg/kg per injection) was highly toxic and it was impossible to evaluate antitumour activity as gemcitabine was toxic at all tested doses. On the contrary and to our great surprise, a dose of elaidic acid (5′)-gemcitabine ester (40 mg/kg) that was shown to be highly active after intraperitoneal administration was also highly active and tolerable when given orally. These results are shown in Table 3.

This surprising finding has been confirmed by the data shown in Table 4, where it is demonstrated that oral administration of elaidic acid (5′)-gemcitabine gives high antitumour activity at tolerable doses with different dosing schedules.

TABLE 3
Antitumour activity in NCR: nu/nu female mice implanted with
Colon 6044 (human colon carcinoma) treated orally with elaidic
acid (5′)-gemcitabine ester or gemcitabine

| | | | | | | BWC | |
|---|---|---|---|---|---|---|---|
| | No. | Treatment | | Dose | Toxic | [%] | Optimum T/C |
| Compound | mice | days | Route | mg/kg | deaths (d) | D 13 | [%] (on day) |
| Saline | 8 | Q3 × 5 | Oral | | | −2 | |
| Elaidic acid (5′)- | 8 | Q3 × 5 | Oral | 40 | 2/8 (15) | −7 | 5(27)* |
| gemcitabine ester | | | | | | | |
| Elaidic acid (5′)- | 8 | Q3 × 5 | Oral | 60 | 6/8(12-24) | −9 | Toxic |
| gemcitabine ester | | | | | | | |
| Elaidic acid (5′)- | 8 | Q3 × 5 | Oral | 80 | 6/8(16-22) | −6 | Toxic |
| gemcitabine ester | | | | | | | |
| Gemcitabine | 8 | Q3 × 5 | Oral | 120 | 7/8(11-16) | −16 | Toxic |
| Gemcitabine | 8 | Q3 × 5 | Oral | 180 | 7/8(11-16) | −22 | Toxic |
| Gemcitabine | 8 | Q3 × 5 | Oral | 240 | 8/8(11-15) | −21 | Toxic |

*significant different from Saline control

Antitumour Activity of Elaidic Acid (5′)-Gemcitabine Ester in Co6044 After Oral Administration

TABLE 4
Antitumour activity in NCR: nu/nu female mice implanted with Colon 6044 (human
colon carcinoma) treated orally with elaidic acid (5′)-gemcitabine ester

| | No. | | | Dose | Toxic | BWC | Optimum T/C |
|---|---|---|---|---|---|---|---|
| Compound | mice | Treatment days | Route | mg/kg | deaths (d) | [%] | [%] (on day) |
| Saline | 7 | D 7-11 | Oral | | | −1 | |
| Elaidic acid (5′)- | 7 | D 7, 14 | Oral | 100 | 1(20) | −4 | 4(24)* |
| gemcitabine ester | | | | | | | |
| Elaidic acid (5′)- | 7 | D 7, 14 | Oral | 50 | 0 | 0 | 22(17)* |
| gemcitabine ester | | | | | | | |
| Elaidic acid (5′)- | 7 | D 7, 10, 13, 16, 19 | Oral | 20 | 1(17) | −3 | 16(24)* |
| gemcitabine ester | | | | | | | |
| Elaidic acid (5′)- | 7 | D 7, 10, 13, 16, 19 | Oral | 15 | 0 | −1 | 27(24)* |
| gemcitabine ester | | | | | | | |
| Elaidic acid (5′)- | 7 | D 7, 10, 13, 16, 19 | Oral | 10 | 0 | −1 | 35(24)* |
| gemcitabine ester | | | | | | | |
| Elaidic acid (5′)- | 7 | D 7-11 | Oral | 10 | 0 | −5 | 8(17)* |
| gemcitabine ester | | | | | | | |
| Elaidic acid (5′)- | 7 | D 7-11 | Oral | 5 | 0 | −3 | 10(28)* |
| gemcitabine ester | | | | | | | |

*significant different from Saline control

High dose dependent activity was seen in all tested schedules after oral administration of elaidic acid (5′)-gemcitabine ester. Significant antitumour activity was observed for all the tested schedules.

1-9. (canceled)

10. A method of treating cancer in a human patient comprising orally administering to the patient a therapeutically effective amount of a gemcitabine derivative of formula I: 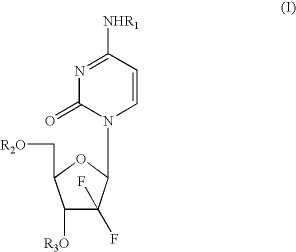
wherein R₁, R₂ and R₃ are independently selected from hydrogen and C₁₈— and C₂₀-saturated and monosaturated acyl groups, with the proviso that R₁, R₂ and R₃ cannot all be hydrogen, or a pharmaceutically acceptable salt thereof.

11. The method of claim 10, wherein the gemcitabine derivative is administered at a rate of about 0.1 mg to 20 grams per day.

12. The method of claim 10, wherein the gemcitabine derivative is administered at a rate of about 100 mg to 2 grams per day.

13. The method of claim 10, wherein the gemcitabine derivative is elaidic acid (5′)-gemcitabine ester.

14. The method of claim 13, wherein the elaidic acid (5′)-gemcitabine ester is administered in unsalified form.

15. The method of any of claims 10-14, wherein the gemcitabine derivative is in admixture with at least one member of the group consisting of pharmaceutically acceptable excipients, diluents, and carriers.

16. A pharmaceutical composition in oral dosage form, in which the composition comprises a gemcitabine derivative of formula I: 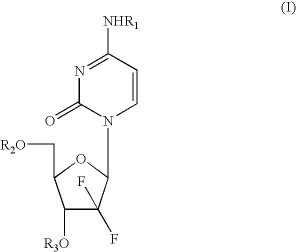
wherein R₁, R₂ and R₃ are independently selected from hydrogen and C₁₈— and C₂₀-saturated and monosaturated acyl groups, with the proviso that R₁, R₂ and R₃ cannot all be hydrogen, or a pharmaceutically acceptable salt thereof.

17. The composition of claim 16, wherein each dosage unit contains about 0.1 mg to 20 grams of the gemcitabine derivative.

18. The composition of claim 16, wherein each dosage unit contains about 100 mg to 2 grams of the gemcitabine derivative.

19. The composition of claim 16, wherein the gemcitabine derivative is elaidic acid (5′)-gemcitabine ester.

20. The composition of claim 19, wherein the elaidic acid (5′)-gemcitabine ester is in unsalified form.

21. The composition of any of claims 16-20, wherein the gemcitabine derivative is in admixture with at least one member of the group consisting of pharmaceutically acceptable excipients, diluents, and carriers.
